(19) United States
(12) Patent Application Publication
(10) Pub. No.: US 2025/0189541 A1
(43) Pub. Date: Jun. 12, 2025

(54) PROTEIN BIOMARKERS OF INFLAMMATORY ACTIVITY IN MULTIPLE SCLEROSIS

(71) Applicants: Oklahoma Medical Research Foundation, Oklahoma City, OK (US); The Board of Regents of the University of Oklahoma, Norman, OK (US)

(72) Inventors: Robert C. AXTELL, Oklahoma City, OK (US); Saurabh GAWDE, Oklahoma City, OK (US)

(73) Assignees: Oklahoma Medical Research Foundation, Oklahoma City, OK (US); The Board of Regents of the University of Oklahoma, Norman, OK (US)

(21) Appl. No.: 18/840,269

(22) PCT Filed: Feb. 3, 2023
(86) PCT No.: PCT/US2023/061937

§ 371 Date: Aug. 21, 2024

Related U.S. Application Data
(60) Provisional application No. 63/312,168, filed on Feb. 21, 2022.

(51) Int. Cl. G01N 33/68; A61K 45/06; C12Q 1/6883

(52) U.S. Cl. CPC G01N 33/6896; A61K 45/06; C12Q 1/6883; C12Q 2600/112; C12Q 2600/158; G01N 2333/96455; G01N 2333/9723; G01N 2800/285; G01N 2800/54

(57) ABSTRACT

The present invention includes a method of treating a subject having relapsing-remitting multiple sclerosis (RRMS) that is undergoing relapse, comprising: determining a level of expression for two or more biomarkers selected from urokinase plasminogen activator (uPA), kallikrein-8 (hK8), kallikrein-11 (hK11), or desmoglein-3 (DSG3) in a biological sample of a subject when compared to the same type of sample from a subject or a population of subjects that do not have RRMS; diagnosing that the subject in undergoing relapse if the level of expression of the two or more biomarkers has decreased in the subject; and administering a therapeutically effective amount of treatment for RRMS to the subject.

### CROSS-REFERENCE TO RELATED APPLICATIONS

This application claims priority to U.S. Provisional Application Ser. No. 63/312,168, filed Feb. 21, 2022, the entire contents of which is incorporated herein by reference.

### STATEMENT OF FEDERALLY-FUNDED RESEARCH

This invention was made with government support under R01AI137047 and R01EY027346 awarded by the National Institutes of Health. The government has certain rights in the invention.

### TECHNICAL FIELD OF THE INVENTION

The present invention relates in general to the field of novel protein biomarkers, and more particularly, to novel protein biomarkers of inflammatory activity in multiple sclerosis (MS).

### BACKGROUND OF THE INVENTION

Without limiting the scope of the invention, its background is described in connection with multiple sclerosis (MS).

Multiple sclerosis (MS) is a chronic inflammatory disease of the central nervous system (CNS) that afflicts over 2.5 million people around the world. It is thought to be initiated by an autoimmune response to CNS antigens which leads to the destruction of myelin and neurons and renders patients disabled [1]. Approximately 85% of MS patients are diagnosed with an initial relapsing and remitting disease course. Relapses (MS flare or exacerbations) are defined as new or worsening clinical symptoms which are then followed by periods of remission where patients have partial or full recovery. Disability accumulates when patients do not fully regain normal function. It is often difficult to determine whether a patient is experiencing a true relapse or a pseudo-relapse confounded by infection and comorbidities [2]. Gadolinium MRI contrast scans are often used to validate a relapse [3]. However, MRIs are expensive and may miss lesion location. There are emerging toxicity concerns with repeated gadolinium exposure [4]. In many instances, MRIs are used when clinical signs are already evident and inflammatory damage has occurred. Furthermore, CSF oligoclonal bands and the IgG index are helpful in diagnosis but not for predicting relapses and progression (Becker M et al). Therefore, an inexpensive non-invasive method for detecting relapses is much in need and it will benefit both clinical trial and clinical practice.

Neurofilaments are cytoskeletal proteins released from damaged axons into the cerebrospinal fluid (CSF) and the blood. Recent technology allows serum neurofilament light chain (sNfL) be detected and shown to be elevated in MS patients with impending or recent clinical relapse [5-8]. However, elevated sNfL are also associated with other neurodegenerative diseases and increased age in a healthy population [9-11]. This raises the question about specificity of sNfL as a marker for MS relapse. Additionally, reliable detectability of NfL levels in blood is a concern. Only a small fraction of sNfL that leaks from the damaged CNS is detected in the blood. Therefore, minor disease-relevant fluctuations in sNfL levels may not be detected even with the most sensitive single molecular array (SiMoA) technology, questioning the sensitivity of using only sNfL as a blood-based biomarker for MS [12].

Relapsing-remitting multiple sclerosis (RRMS) is characterized by episodes of new or worsening clinical symptoms followed by recovery. There is need for biomarker development beyond clinical manifestations and MRI. Serum neurofilament light chain (sNfL) has emerged as a biomarker for inflammatory activity in MS. However, there are limitations to the sensitivity and specificity of sNfL in identifying relapses.

Thus, what is needed are biomarkers that distinguish patients having a relapse compared to a pseudo-relapse, and/or biomarkers that can be used to monitor treatment response in a clinical trial setting as a surrogate for MRI.

### SUMMARY OF THE INVENTION

As embodied and broadly described herein, an aspect of the present disclosure relates to a method of treating a subject having relapsing-remitting multiple sclerosis (RRMS) that is undergoing relapse, comprising: (a) determining a level of expression for two or more biomarkers selected from urokinase plasminogen activator (uPA), kallikrein-8 (hK8), kallikrein-11 (hK11), or desmoglein-3 (DSG3) in a biological sample of a subject when compared to the same type of sample from a subject or a population of subjects that do not have RRMS; (b) diagnosing the subject in (a) as undergoing relapse if the level of expression of the two or more biomarkers has decreased in the subject; and (c) administering a therapeutically effective amount of a treatment for RRMS to the subject diagnosed in (b). In one aspect, the biomarkers are uPA, hK8, hK11, or DSG3. In another aspect, the method further comprises determining the level of expression for NfL, uPA, hK8, hK11, or DSG3, wherein a combination of biomarkers reaches a higher area under the curve when compared to NfL alone. In another aspect, the therapeutic agent is selected from at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH) or ponesimod. In another aspect, the biological sample is selected from the group consisting of peripheral blood, plasma, serum, peripheral blood mononuclear cells (PBMCs), or cerebrospinal fluid (CSF). In another aspect, the relapse is acute phase relapse. In another aspect, the relapse is from a patient that had previously received at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod. In another aspect, the biomarkers are nucleic acid biomarkers, protein biomarkers, or combinations thereof. In another aspect, the method further comprises detecting in a sample from a subject with RRMS previously treated with a steroid treatment an increase in a level of expression of FK506 binding protein 1 (FKBP5), wherein cessation of steroid treatment causes a decrease in the level of expression of FKBP5.

As embodied and broadly described herein, an aspect of the present disclosure relates to a method for determining whether a subject with relapsing-remitting multiple sclerosis (RRMS) with an elevated level of an NfL biomarker is having or will have a relapse, comprising: (a) determining in a biological sample from the subject a level of expression for one or more biomarkers selected from uPA, hK8, hK11, or DSG3 when compared to the same sample from a subject or a population of subjects that do not have RRMS, wherein the combination of the NfL biomarker and the one or more biomarker selected from uPA, hK8, hK11, or DSG3 has a higher sensitivity and selectivity that using NfL biomarker alone; (b) diagnosing the subject in (a) as undergoing relapse if the expression level of the uPA, hK8, hK11 or DSG3 has decreased, and (c) administering a therapeutically effective amount of a treatment for RRMS to the subject diagnosed in (b). In one aspect, the method comprises selecting 2, 3, or 4 of the biomarkers selected from uPA, hK8, hK11, or DSG3. In another aspect, the method of claim 10, further comprising determining the level of expression for NfL, uPA, hK8, hK11, or DSG3 and calculating an area under the curve, wherein a combination of biomarkers reaches an area under the curve of at least 0.87. In another aspect, the therapeutic agent is selected from at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod. In another aspect, the biological sample is selected from the group consisting of peripheral blood, plasma, serum, peripheral blood mononuclear cells (PBMCs) or CSF. In another aspect, the relapse is acute phase relapse. In another aspect, the relapse is from a patient that had previously received at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod. In another aspect, the biomarkers are nucleic acid biomarkers, protein biomarkers, or combinations thereof. In another aspect, the method further comprises detecting in a sample from a subject with RRMS previously treated with a steroid treatment an increase in a level of expression of FK506 binding protein 1 (FKBP5), wherein cessation of steroid treatment causes a decrease in the level of expression of FKBP5.

As embodied and broadly described herein, an aspect of the present disclosure relates to a method for detecting relapse in a subject with relapsing-remitting multiple sclerosis (RRMS), comprising: (a) determining a level of expression for two or more biomarkers selected from uPA, hK8, hK11, or DSG3 in a biological sample of the subject when compared to the same sample from a subject or a population of subjects that do not have RRMS and wherein one of the biomarkers is not an NfL biomarker, wherein the two or more biomarker selected from uPA, hK8, hK11, or DSG3 has a higher sensitivity and selectivity that the NfL biomarker alone; (b) diagnosing the subject in (a) as undergoing relapse if the level of expression of the two or more biomarkers has decreased in the subject, and (c) administering a therapeutically effective amount of a treatment for RRMS to the subject diagnosed in (b). In one aspect, the method comprises selecting 2, 3, or 4 of the biomarkers selected from uPA, hK8, hK11, or DSG3. In another aspect, the therapeutic agent is selected from at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod. In another aspect, the biological sample is selected from the group consisting of peripheral blood, plasma, serum, peripheral blood mononuclear cells (PBMCs), or CSF. In another aspect, the relapse is acute phase relapse. In another aspect, the relapse is from a patient that had previously received at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod, and selecting a new therapy. In another aspect, the biomarkers are nucleic acid biomarkers, protein biomarkers, or combinations thereof. In another aspect, the sample from the subject with RRMS has been previously detected to have a higher level of NfL expression as compared to the sample from subjects that do not have RRMS. In another aspect, the method further comprises detecting in a sample from a subject with RRMS previously treated with a steroid treatment an increase in a level of expression of FK506 binding protein 1 (FKBP5), wherein cessation of steroid treatment causes a decrease in the level of expression of FKBP5.

### BRIEF DESCRIPTION OF THE DRAWINGS

For a more complete understanding of the features and advantages of the present invention, reference is now made to the detailed description of the invention along with the accompanying figures and in which:

FIGS. 1A to 1F show Quality Control (QC) of Proximity Extension Assay (PEA). Expression levels of 724 serum proteins were assessed across two independent runs (Run 1 and Run 2). (FIG. 1A) Pearson's correlation coefficient (r) was determined for all 724 protein (FIG. 1B) Table showing serum proteins belonging to each group defined by the range of r. Correlation plots for Group 1 serum proteins NFL (FIG. 1C) and Notch 3 (FIG. 1D), Group 2 serum protein IL-7R (FIG. 1E) and Group 3 serum protein IL2 (FIG. 1F) are shown.

FIGS. 2A to 2D show serum protein profiles distinguish RRMS patients from healthy controls. (FIG. 2A) Serum protein profiles of RRMS (n=64) and Healthy controls (n=20) were compared. Significant proteins were determined using unpaired t-tests with multiple comparisons corrected by Bonferroni and Hochberg method setting a False Discovery Rate (FDR) of 5%. (FIG. 2B) PCA analysis of significantly different serum proteins between RRMS and Healthy controls was performed. Gene Ontology (GO) analysis of serum proteins (FIG. 2C) elevated in RRMS and (FIG. 2D) Healthy controls was also performed.

FIGS. 3A to 3C show serum protein profiles distinguish relapse and remission in MS patients. (FIG. 3A) Serum protein profiles of Relapse (n=24) and remitting (n=40) MS patients were compared. Significant proteins were determined using unpaired t-tests with multiple comparisons setting a p-value threshold of ≤0.05. (FIG. 3B) Unsupervised hierarchical cluster analysis of significantly different proteins between patients in relapse and remission classified into patients into two groups, Group 1 and Group 2. (FIG. 3C) Pie charts depict the frequency of patients in relapse or remission in Group 1 and Group 2. Chi-squared test showed statistical significance between Group 1 and Group 2 p<0.0001.

FIGS. 4A to 4I show that steroids alter serum protein profiles of MS Relapse patients (FIG. 4A) Classification of RRMS patients based on disease status and steroids. Serum protein profiles of (FIG. 4B) MS relapse patients on steroid treatment and MS remission patients, (FIG. 4C) MS relapse patients without steroid treatment and MS remission patients and (FIG. 4D) MS relapse patients on steroid treatment and MS relapse patients without steroid treatment were compared. Significant proteins were determined using unpaired t-tests with multiple comparisons setting a p-value threshold of ≤0.05. (FIG. 4E) Venn diagram of differentially abundant serum proteins of relapse steroids vs remission and relapse steroids vs relapse no steroids comparisons. Serum protein levels of (FIG. 4F) FKBP5 and (FIG. 4H) NFL were compared between MS relapse patients on steroids, MS relapse patients without steroids and MS remission patients. P-values were determined using one-way ANOVA and p<0.05 was significant. Serum protein levels of (FIG. 4G) FKBP5 and (FIG. 4I) NFL were correlated with weeks after steroids in MS relapse patients. p-values<0.05 were considered significant.

FIGS. 5A to 5C show the results from monitoring longitudinal changes in serum proteins from relapse to remission. (FIG. 5A) Fold Change of serum proteins that were significantly different between MS relapse and remission. Significant proteins were determined using paired t-tests with multiple comparisons setting a p-value threshold of ≤0.05. (FIG. 5B) Serum levels of NFL were compared between MS patients under remission, MS relapse patients with no steroid treatment and MS relapse patients with steroid treatment using ordinary one-way ANOVA. P-values<0.05 were considered significant (FIG. 5C) Regression of NFL serum levels with weeks after relapse in MS Relapse. P-values<0.05 were considered significant.

FIGS. 6A to 6D show that a panel of serum proteins classifies MS relapse with higher accuracy than NFL alone. (FIG. 6A) Venn diagram comparing significantly abundant proteins between MS relapse and remission from OMRF and Stanford cohort. (FIG. 6B) Table shows the six serum analytes that are significantly different between Relapse and Remission in OMRF and Stanford cohorts. (FIG. 6C) Receiver Operating Characteristics (ROC) curves examining the predictive performance of serum protein biomarkers for identifying MS relapse in the longitudinal cohort. (FIG. 6D) Table showing AUC scores for all the regression models.

FIG. 7 is a graph that shows the AUCs for the various combinations.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present inventors used a large-scale, non-targeted multiplex proteomics assay to discover relapse-associated protein markers in MS patients. The inventors identified novel proteins that are associated with relapse in MS patients and determined that a panel of protein markers increased sensitivity and specificity in identifying relapses compared to using sNfL as the sole marker.

More particularly, the inventors used a multiplex assay to measure levels of blood proteins in two distinct RRMS patient cohorts. The first was a cross-sectional cohort of 64 patients and 20 healthy controls. The second was a longitudinal cohort of 12 patients. Levels of 724 proteins were assessed in both cohorts using proximity extension assay (PEA) from Olink Biosciences. Blood protein profiling was performed using multiple paired and unpaired t-tests with age and sex covariate correction. Logistic regression models to identify relapsing patients were generated and Area under the Curve (AUC) was used as a comparative metric.

In the cross-sectional cohort, the inventors identified 5 proteins, including NfL, that were elevated and 41 proteins that were reduced in patients experiencing a relapse. In the longitudinal study, the inventors found that 10 proteins, including NfL, were elevated and 26 proteins were reduced in patients with a relapse. Next, the inventors compared the differentially abundant proteins in relapse and remission in the cross-sectional and longitudinal cohorts and identified 5 proteins that were common between those relapsing and those that were not. Finally, using logistic regression analysis to generate Receiver Operating Characteristics (ROC) curves, the inventors found that the 5 common proteins could identify relapse in patients with higher accuracy (AUC=0.87) than sNfL alone (AUC=0.71).

Using two distinct cohorts of RRMS patients, these studies confirmed that sNfL levels were elevated during relapse. Furthermore, the inventors identified 4 novel blood proteins that are differentially abundant during a relapse. Together, these blood biomarkers can be used as a panel to monitor disease activity in RRMS patients.

As used herein, a “biological sample” refers to a biological sample derived from a bodily fluid, such as blood, preferably peripheral (or circulating) blood or cerebrospinal fluid. A blood sample may be, e.g., whole blood, serum or plasma. In certain embodiments, serum may be used as the source for the biomarkers as the samples are readily available and often obtained for other sampling, is stable, and requires less processing, thus making it ideal for locations with little to refrigeration or electricity, is easily transportable, and is commonly handled by medical support staff.

In certain embodiments, the expression level of the selected biomarkers are measured in a blood, plasma, serum, or cerebrospinal fluid sample obtained from the subject. In some embodiments, the expression level of the one or more biomarkers is compared to the expression level of the corresponding one or more biomarkers in a statistical sample representative of the subject, wherein the comparison is used to determine if the subject warrants diagnostic screening for Relapse-Remission of Multiple Sclerosis (RRMS).

As used herein, a “normal” individual or a sample from a “normal” individual refers to quantitative data, qualitative data, or both from an individual who has or would be assessed by a physician as not having Relapse-Remission of Multiple Sclerosis (RRMS). Often, a “normal” individual is also age-matched within a range of 1, 2, 3, 4, 5, 6, 7, 8, 9, or 10 years with the sample of the individual to be assessed.

As used herein, the term “treatment” refers to the alleviation, amelioration, and/or stabilization of symptoms, as well as delay in progression of symptoms of a particular disorder, specifically, Relapse-Remission of Multiple Sclerosis (RRMS). For example, “treatment” of Multiple Sclerosis (MS) includes any one or more of: (1) elimination of one or more symptoms of MS, (2) reduction of one or more symptoms of MS, (3) stabilization of the symptoms of MS (e.g., failure to progress to more advanced stages of MS), and (4) delay in onset of one or more symptoms of MS; and (5) delay in progression (i.e., worsening) of one or more symptoms of MS. Treatments for MS for use with the present invention include providing the patient suspected of RRMS based on the detection of the biomarkers herein with a therapeutic agent selected from at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod.

In some embodiments, the one or more biomarkers comprise uPA, hK8, hK11, or DSG3. In some embodiments, the two or more biomarkers comprise uPA and hK8. In some embodiments, the three or more biomarkers comprise uPA, hK8, and hK11. In some embodiments, the four biomarkers comprise uPA, hK8, hK11, and DSG37. In some embodiments, the five biomarkers comprise uPA, hK8, hK11, DSG3, and NfL. In some embodiments, the four or more biomarkers comprise hK8, hK11, DSG3, and NfL. In some embodiments, the three or more biomarkers comprise hK11, DSG3, and NfL. In some embodiments, the two or more biomarkers comprise DSG3 and NfL. In some embodiments, the one or more biomarkers comprise uPA, hK11, DSG3, and NfL. In some embodiments, the one or more biomarkers comprise uPA, hK8, DSG3, and NfL. In some embodiments, the one or more biomarkers comprise uPA, hK8, hK11, and NfL. In some embodiments, the one or more biomarkers comprise uPA, DSG3, and NfL. In some embodiments, the one or more biomarkers comprise hK8, hK11, and NfL. In some embodiments, the one or more biomarkers comprise uPA, hK11, DSG3, and NfL. In some embodiments, the one or more biomarkers comprise uPA, hK8, DSG3, and NfL. In some embodiments, the one or more biomarkers comprise hK8, hK11, and DSG3. In some embodiments, the one or more biomarkers comprise uPA, hK11, and DSG3. In some embodiments, the one or more biomarkers comprise uPA, hK8, and DSG3. In some embodiments, the one or more biomarkers comprise uPA, hK8, and hK11. In some embodiments, the one or more biomarkers comprise uPA and DSG3. In some embodiments, the one or more biomarkers comprise hK8 and DSG3. In some embodiments, the one or more biomarkers comprise hK11 and DSG3.

Patient Cohorts. Cross-sectional cohort: Serum samples were collected from 64 RRMS patients and 20 healthy donors at the Oklahoma Medical Research Foundation (OMRF) Multiple Sclerosis Center of Excellence. Demographic information for OMRF MS patients and healthy controls is shown in Supplemental Table 1. MS relapse was defined as new or worsening clinical symptoms lasted more than 48 hours in absence of fever, infection or other confounding medical conditions. It was further determined using both clinical assessment as well as presence of increased gadolinium enhanced lesions using magnetic resonance imaging (MRI) [13]. All serum samples were obtained before the initiation of disease-modifying therapies (DMTs).

Longitudinal cohort: Paired plasma samples were collected from 12 RRMS patients from the Stanford MS Clinic. Demographic and treatment information for Stanford RRMS patients is shown in Supplemental Table 2. Samples were collected during clinical relapse and remission (between 0 to 19 months) from these patients.

For both cohorts, patients were diagnosed with RRMS using the McDonald criteria by a board-certified neurologist. Written informed consent was obtained from individuals prior to participation in the study, which was approved by the Oklahoma Medical Research Foundation's Institutional Review Board. Assays on the samples were performed blinded from the clinical data.

Protein Quantification. Proteins were measured with the Olink Cardiometabolic, Cardiovascular III, Development, Inflammation, Neuro-exploratory, Neurology, Oncology II and Oncology III panels using proximity extension assay (PEA) technology [14]. In total, the inventors used a multiplex assay to measure levels of blood proteins in two distinct RRMS patient cohorts. The first was a cross-sectional cohort of 64 patients and 20 healthy controls. The second was a longitudinal cohort of 12 patients. Levels of 724 proteins were assessed in both cohorts using proximity extension assay (PEA) from Olink Biosciences. Blood protein profiling was performed using multiple paired and unpaired t-tests with age and sex covariate correction. Logistic regression models to identify relapsing patients were generated and Area under the Curve (AUC) was used as a comparative metric. measured expression levels of 724 proteins. Data are presented as log base-2 normalized protein expression (NPX) values. For the quality-control of PEA [14], expression levels of all 724 serum proteins were measured from two independent runs of 20 individuals from the OMRF cohort and were further correlated across the two runs.

Statistical Analysis. Pearson's correlation coefficient (r) analysis was performed using cor function from stats v4.0.5 in R to test the correlation between NPX values for 724 proteins across two independent runs. Proteins with Pearson's coefficient r>0.75 were considered strongly correlated; 0.5<r<0.75 were considered moderate; and −0.5<r<0.5 were considered weak. Significantly different proteins were determined using multiple t-tests corrected by the Bonferroni and Hochberg method with a False Discovery Rate (FDR) of 5% using limma v3.46 in the Bioconductor suite in R. Principal Components Analysis (PCA) was performed using prcomp function in R and principal components with the highest variation-PCI and PC2-were plotted against each other. Gene Ontology analysis was performed using the STRING database for functional annotation of the significantly different proteins.

Logistic regression analysis was performed using caret v6.0-88 in R to generate univariate or multivariate predictive models that estimate the probability of identifying relapse and remission in RRMS patients. Repeated 10-fold cross-validation was used to correct for the bias arising from validating models on the same dataset used to generate the models. Receiver Operating Characteristics (ROC) curves were generated using the probabilities from each model and the efficacy of each model for classifying relapse and remission in MS patients was evaluated using MLeval v0.3 in R [19]. Area under the curve for the ROC Curve (AUC-ROC) was used to determine the classification accuracy for each model. All these statistical analyses were performed in R (3.6.2, 4.0.5) [15].

Individual serum protein levels were compared using unpaired t-tests with a significance of p<0.05. Serum proteins that were significantly different between paired relapse and remission in the longitudinal cohort were determined using multiple t-tests with a significance of p<0.05. NfL and FKBP5 NPX values were correlated to weeks after cessation of steroids treatment using linear regression.

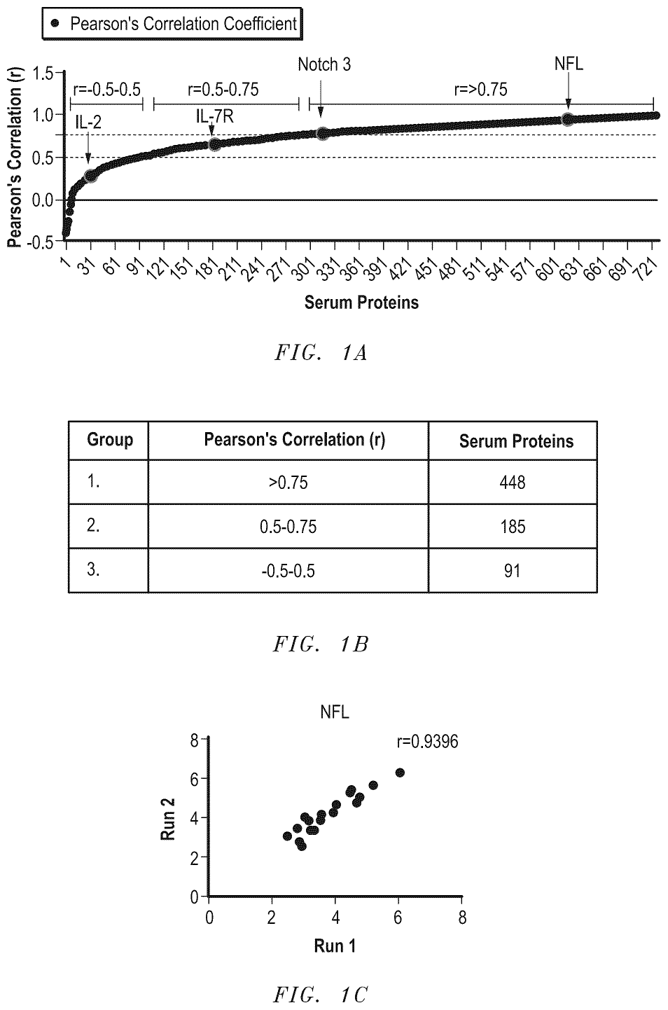

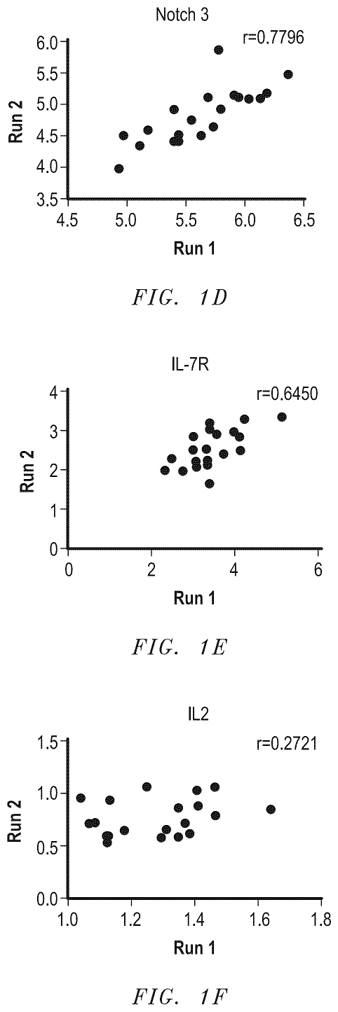

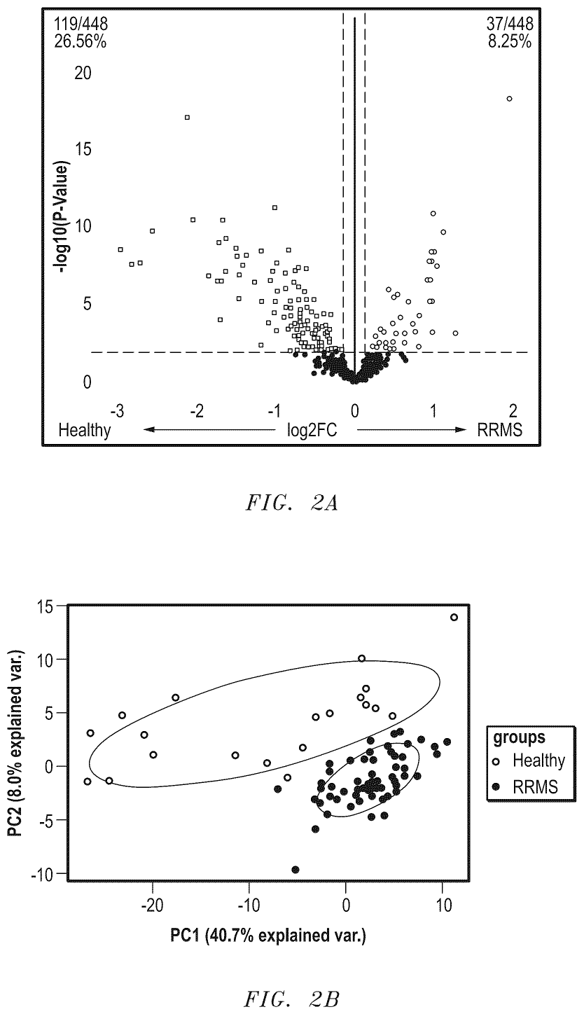

Quality Control of Proximity Extension Assay (PEA), a multiplex protein biomarker assay. Measuring serum proteins using a multiplex approach has been widely used in many diseases including MS [20-22]. Accuracy and reproducibility of the serum proteins measurement is paramount for the discovery of robust biomarkers. In this study, the inventors used a multiplex assay to measure levels of blood proteins in two distinct RRMS patient cohorts. The first was a cross-sectional cohort of 64 patients and 20 healthy controls. The second was a longitudinal cohort of 12 patients. Levels of 724 proteins were assessed in both cohorts using proximity extension assay (PEA) from Olink Biosciences. Blood protein profiling was performed using multiple paired and unpaired t-tests with age and sex covariate correction. Logistic regression models to identify relapsing patients were generated and Area under the Curve (AUC) was used as a comparative metric. used proximity extension assay (PEA) to measure the levels of 724 serum proteins in RRMS patients and healthy controls, with the aim to identify potential biomarkers associated with disease activity in patients. PEA is a relatively new antibody-based analyte detection technology. Therefore, the inventors first determined the reproducibility of each protein measurement in 20 serum samples from the cross-sectional study cohort, which consisted of six healthy donors, eight relapsing patients and 6 remission patients. The inventors performed PEA on these 20 samples in two independent runs and correlated the relative levels of the 724 serum proteins from Run 1 and Run 2 using Pearson's correlation coefficient, r (FIG. 1A).

Based on correlation values, the inventors found that 448 proteins were highly reproducible with r>0.75 (Group 1), 185 proteins were moderately reproducible with r values between 0.5-0.75 (Group 2) and 91 proteins were poorly reproducible with r values between-0.5-0.5 (group 3) (FIG. 1B). Examples of highly reproducible proteins were Neurofilament light chain (NfL) (FIG. 1C) and Notch 3 (FIG. 1D). An example of a moderately reproducible protein was interleukin-7 receptor (IL-7R) (FIG. 1E). Interleukin-2 (IL-2) was an example of a poorly reproducible protein (FIG. 1F). For this study, the inventors considered the 448 proteins with r>0.75 to have passed quality control (QC) and these were used for the subsequent analysis of both the cohorts.

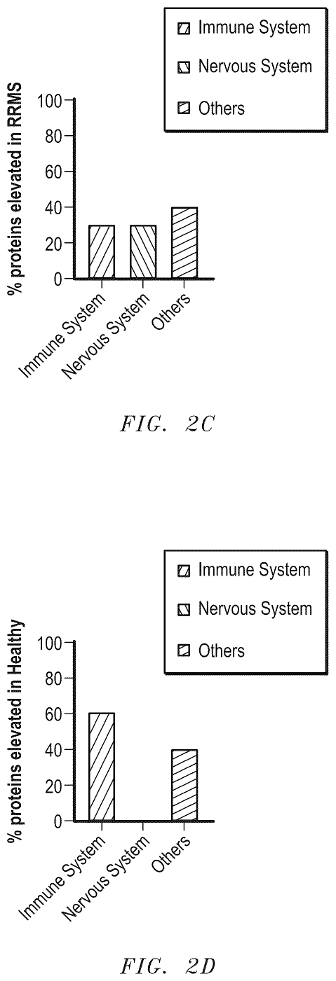

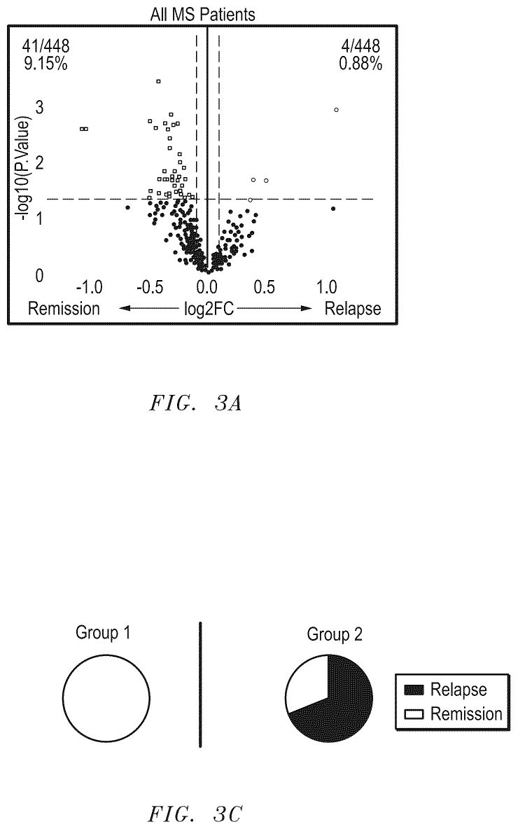

MS patients can be distinguished from healthy donors based on their serum protein signatures. The inventors next determined if serum protein markers can distinguish RRMS patients from healthy donors. For this analysis, the inventors compared levels of the 448 serum proteins that passed QC in RRMS patients and healthy controls using multiple unpaired t-tests setting a False Discovery Rate (FDR) of 5% and adjusted for age and sex as co-variates. The inventors identified that 156 out of 448 proteins were significantly different between RRMS patients and healthy controls. Of these 156 proteins, 37 were elevated and 119 were reduced in RRMS patients compared to healthy controls (FIG. 2A). Next, the inventors performed principal components (PC) analysis to determine if RRMS patients and healthy controls can be distinguished based on the levels of these 156 proteins. Based on PC1 and PC2 which explained 40.7% and 8% of the variance respectively, the inventors could cluster RRMS patients and healthy controls into two distinct groups (FIG. 2B). Gene Ontology (GO) database analysis identified the biological pathways to which these 156 significantly different proteins belonged. Of the 156 proteins, 152 mapped to 8 GO pathways which belonged to immune (GO: 0002376, GO: 0002682, GO: 0006952), nervous (GO: 0007399) and other (GO: 0050789, GO: 0050896, GO: 0048856, GO: 0009987) systems. Of the proteins elevated in RRMS patients, 29.7% mapped to immune system pathways, 29.7% mapped to nervous system pathways and 40.6% mapped to pathways belonging to other systems (FIG. 2C). However, for the proteins reduced in RRMS patients, 59.66% mapped to immune system pathways and 40.34 mapped to pathways belonging to other systems. None of the proteins reduced in RRMS patients compared to healthy controls mapped to nervous system pathways (FIG. 2D). These data highlight the critical involvement of the nervous system in MS pathogenesis and provide evidence that neurological damage in MS can be detected in the serum using the PEA assay.

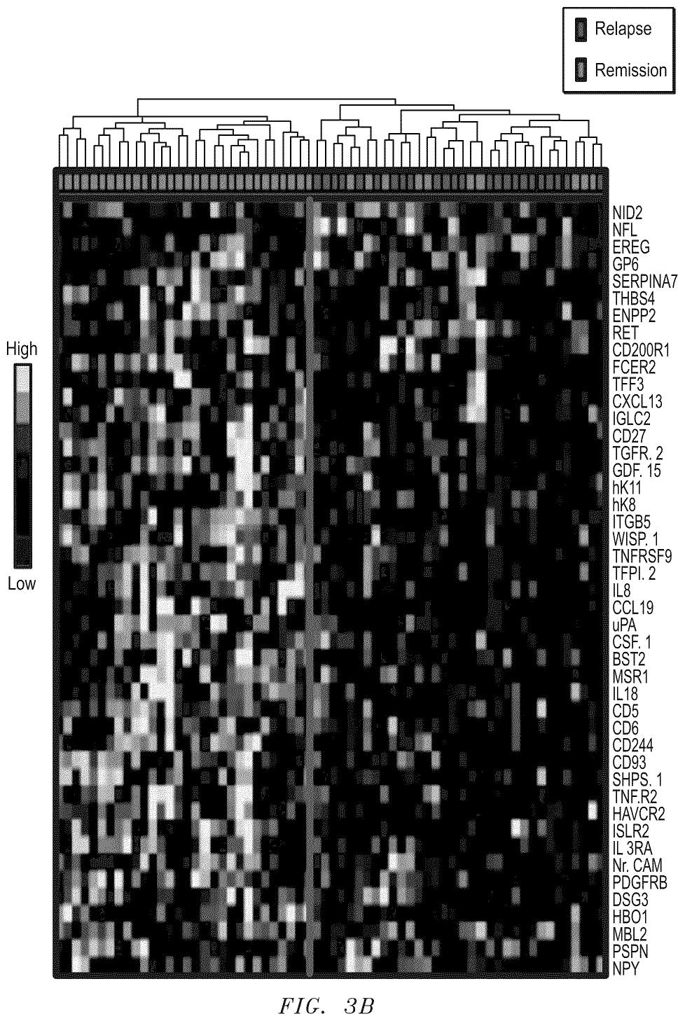

Serum protein signatures can differentiate relapse and remission disease status in MS patients. In the cross-sectional cohort, 24 patients were undergoing a clinical relapse, which was confirmed by active enhancing lesions measured by MRI, and 40 patients were in remission. Using PEA, the inventors identified whether serum protein levels could differentiate between relapse and remission in these patients. The inventors compared levels of the 448 serum proteins (which passed QC) in patients with relapse to the levels in remission using multiple unpaired t-tests setting a p-value threshold of <0.05 and adjusted for age and sex as co-variates. The inventors discovered that 5 proteins were elevated and 41 were reduced in relapsing MS patients compared to remitting MS patients (FIG. 3A). Next, the inventors performed unsupervised hierarchical cluster analysis of all the MS patients based on expression levels of these 45 significantly different proteins. This analysis found that MS patients grouped into two separate groups, Group 1 and Group 2 (FIG. 3B). Group 1 consisted entirely of MS patients in remission. Group 2 consisted of 70% relapsing MS patients and 30% remission MS patients (FIG. 3C). Therefore, these data show that MS patients under clinical relapse or remission can be distinguished based on their serum protein signatures.

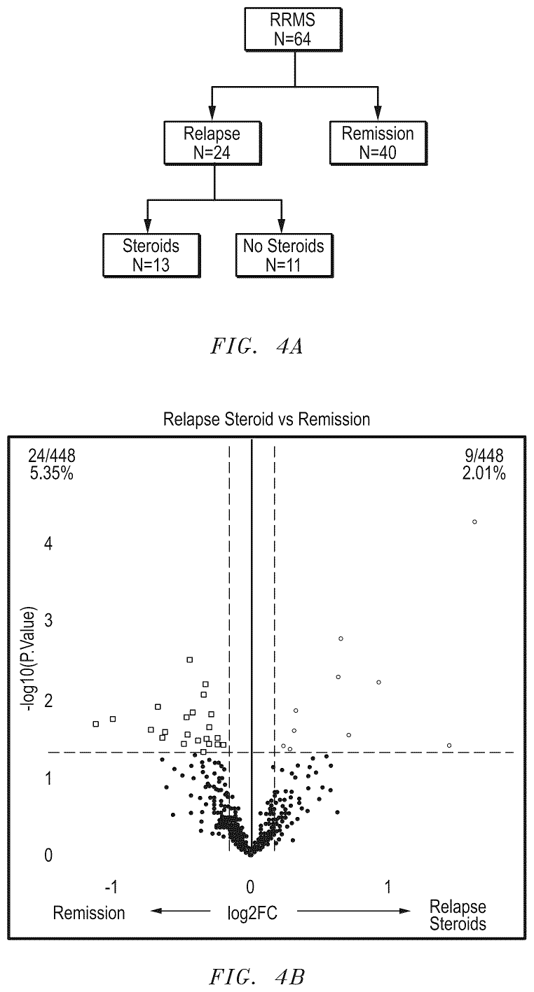

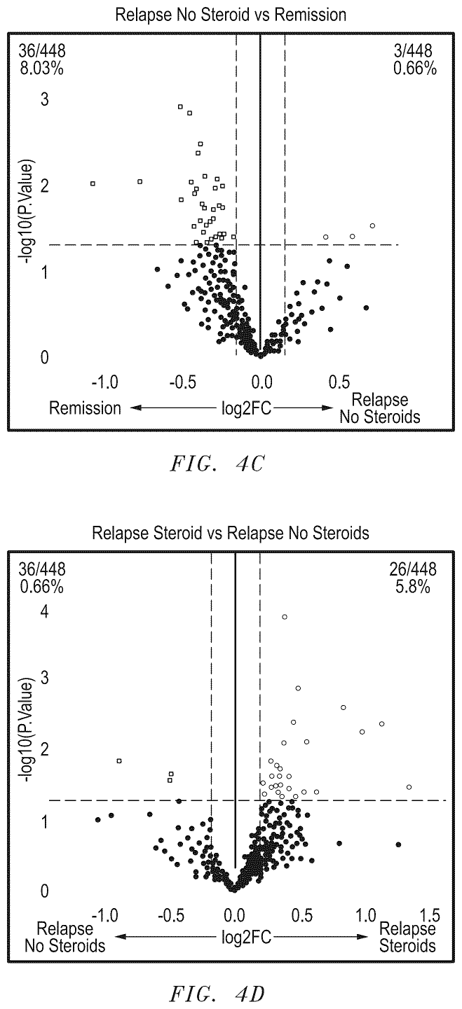

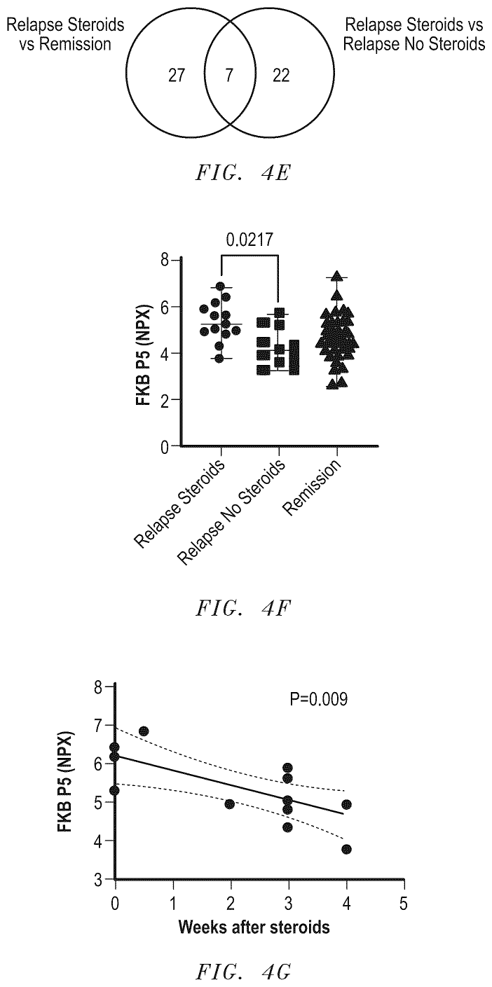

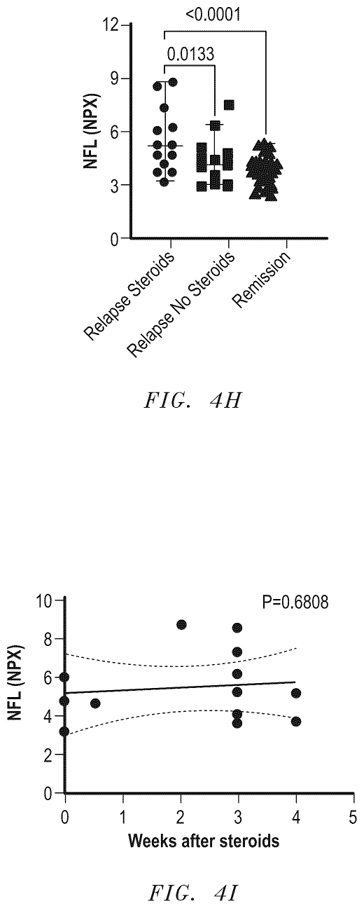

Intravenous steroid treatment alters the serum protein profiles of relapsing MS patients. High dose corticosteroids administered intravenously or orally are short-term medications used to treat clinical relapses in MS patients [23, 24]. Administration of steroids has been shown to reduce inflammation by decreasing migration of autoreactive immune cells into the CNS, inducing apoptosis in autoreactive leukocytes, and reducing production of inflammatory cytokines [25-28]. Therefore, the inventors next determined whether steroids treatment influenced the serum protein profiles of relapsing MS patients. In the cross-sectional cohort, 13 out of 24 relapsing MS patients received high dose of IV and/or oral steroids during or before blood draw while the remaining 11 did not have steroids (FIG. 4A). The inventors performed three comparative analyses, including, compared serum protein levels in (1) relapsing MS patients on steroids vs remission MS patients, (2) relapsing MS patients without steroids vs remission MS patients and (3) relapsing MS patients on steroids vs relapsing MS patients without steroids. A multiple unpaired t-tests with a p-value threshold of <0.05 was used and adjusted for age and sex as co-variates for this analysis. It was found that 9 proteins were elevated, and 24 proteins were reduced in relapsing MS patients on steroids compared to remitting MS patients (FIG. 4B). The inventors also identified that 3 proteins were elevated, and 36 proteins were reduced in relapsing MS patients without steroids compared to remission MS patients (FIG. 4C). Additionally, the inventors determined that 26 proteins were elevated, and 3 proteins were reduced in relapsing MS patients on steroids compared to relapsing MS patients without steroids (FIG. 4D).

The inventors identified proteins with a strong association with steroid treatment in this cohort of relapsing MS patients by examining differentially abundant proteins that were common between the following comparisons: (1) relapsing MS patients on steroids vs remitting MS patients and (2) relapsing MS patients without steroids vs remission MS patients. The inventors observed seven common proteins in this analysis (FIG. 4E). Of the serum proteins that were significantly altered in steroid patients, FK506 binding protein 51 (FKBP5) and NfL were of interest. Expression of FKBP5 is directly increased in response to steroids engaging glucocorticoid receptor on cells [29, 30]. In this cohort of patients, levels of FKBP5 were significantly increased in relapsing MS patients on steroids compared to relapsing MS patients without steroids (FIG. 4F). It was also found that levels of FKBP5 significantly diminished with time after the cessation of steroids treatment in the relapsing MS patients (FIG. 4G). These data show that the biological effects of steroid therapy can be monitored with serum levels of FKBP5. It was also intriguing that NfL levels were significantly increased in relapsing MS patients on steroids compared to both relapsing MS patients without steroids and remitting MS patients (FIG. 4H). However, NfL serum levels did not significantly decrease with time after stoppage of steroids treatment (FIG. 4I), which suggest that the elevated levels of NfL were not a direct effect of steroids treatment in these patients. These data show that the administration of high dose steroids affects serum protein profiles of relapsing MS patients and is a confounder to be considered in biomarker discovery.

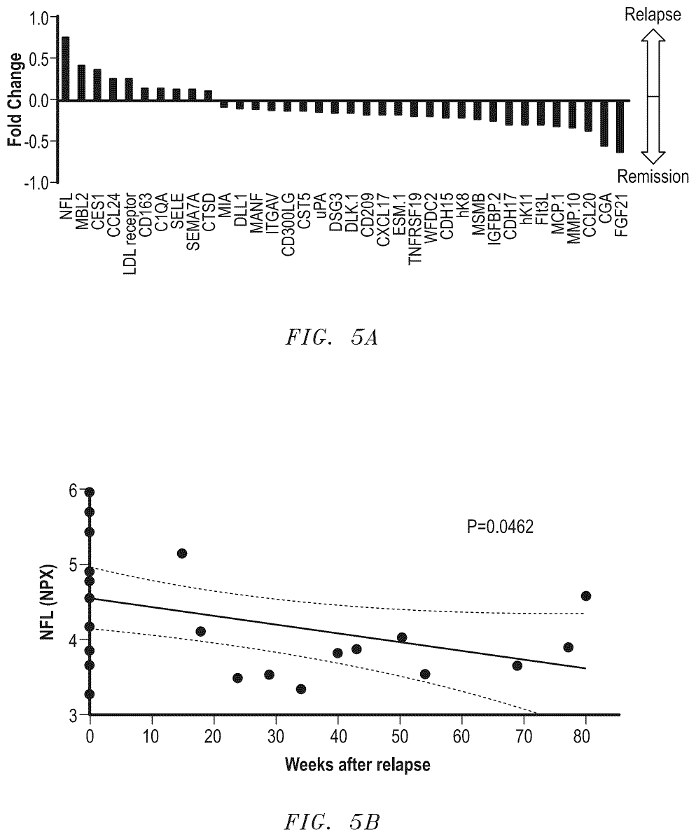

Longitudinal changes in blood protein levels from relapse to remission. To verify these findings from the cross-sectional cohort described above, the inventors assessed blood proteins in a separate longitudinal cohort, where patients had blood draws during a relapse and during remission. The expression level of blood proteins was compared, which passed QC, in longitudinal blood draws during relapse and remission from 12 RRMS patients using multiple paired t-tests with a p-value threshold of <0.05. In this cohort, 10 proteins identified were elevated, and 26 proteins were reduced in relapsing MS patients compared to remission MS patients (FIG. 5A). It is of note that NfL was the most elevated blood protein in relapse and levels of NfL were negatively correlated with the length of time taken from relapse to blood draw (FIG. 5B). In this cohort, only two patients in relapse were on acute steroid therapy at blood draw. NfL levels are not highly abundant in the blood of these relapsing patients on steroids (FIG. 5B). This further demonstrates that steroid treatments do not directly affect serum NfL levels during an active relapse.

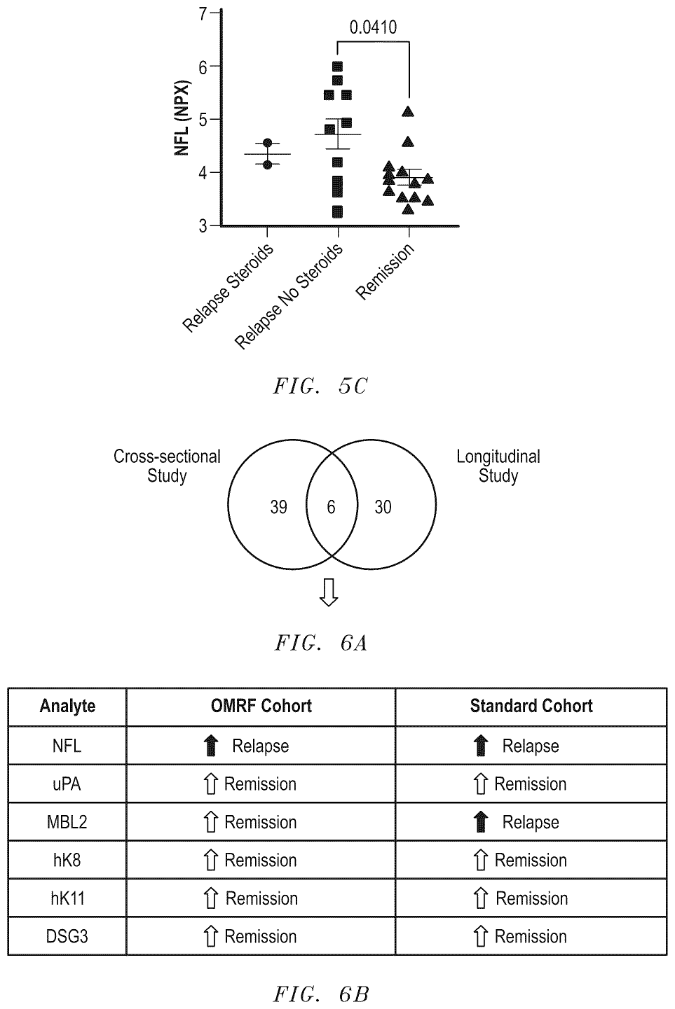

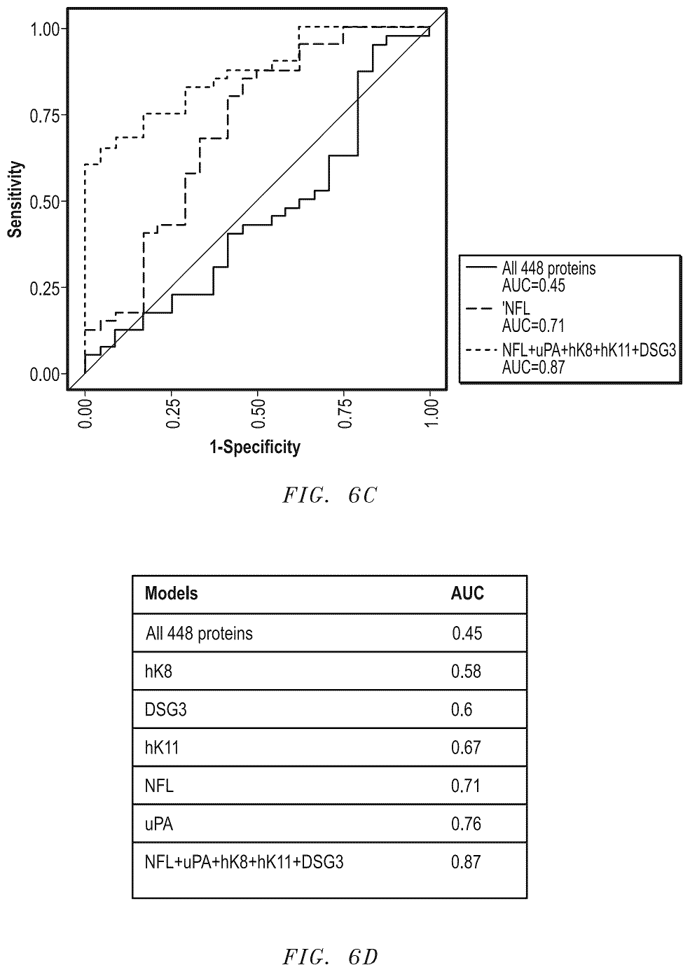

A panel of blood proteins identifies MS relapse with higher accuracy than NfL alone. Next, proteins were identified that were significantly different in relapse and remission that were common in the cross-sectional and the longitudinal study cohort (FIG. 6A). It was found 6 proteins that were common between the two cohorts. NfL was the only protein that was elevated in relapse in both studies. Whereas urokinase plasminogen activator (uPA), kallikrein-8 (hK8), kallikrein-11 (hK11) and desmoglein-3 (DSG3) were all reduced in relapse in both cohorts. Although, mannose binding lectin 2 (MBL2) was significantly different in both cohorts, the inventors found that it was reduced in relapse in the cross-sectional cohort but increased in relapse in the longitudinal cohort (FIG. 6B).

Even though, serum NfL is considered a marker of relapse in MS patients, there are questions about the sensitivity of this serum marker to determine relapses. Therefore, the inventors compared the effectiveness of NfL alone with the panel of 5 serum proteins, verified in both cohorts, to distinguish patients in relapse or in remission. Due to sample size restrictions, this analysis was performed on the cross-sectional cohort. The inventors generated logistic regression models using one or more serum proteins, also called predictor variables, using the cross-sectional cohort. The inventors tested the logistic regression models using repeated 10-fold cross-validation on the cross-sectional cohort and plotted receiver operating characteristic (ROC) curves for each model [31]. It was found that a regression model with a combination of NfL, uPA, hK8, hK11 and DSG3 was able to classify relapse with an area under the curve (AUC) of 0.87 compared to a NfL model alone which had an AUC of 0.71 (FIG. 6C). Models using individual proteins other than NfL were also tested to classifying relapse (FIG. 6D). The models were generated and tested on the same dataset; therefore, they are inherently optimistic. However, the goal was to display that a combination of protein markers outperforms NfL alone in classifying MS relapse, which the results herein show. In summary, these data show that a regression model using a combination of NfL, uPA, hK8, hK11 and DSG3 can classify relapse in MS patients with higher AUC than NfL alone.

Discovery of biomarkers for disease activity is an active area of research in MS. There is emerging interest in measuring NfL levels in the blood to determine if a patient is undergoing an active relapse. However, there are limitations. First, there is a low specificity as other disease states also have elevated sNfL levels. Second, sNfL levels in the serum increase with age. Third, elevated sNfL levels may also be associated disease progression, where there is an increase in disability in the absence of relapses. Finally, though there may be statistical differences in sNfL at group level, sNfL cannot, itself, be solely used to determine relapse in an individual patient. As both sensitivity and specificity of sNfL for relapse is not ideal, sNfL levels can also be included in a panel of other biomarkers to help with predicting relapse. This study used a high throughput proteomic assay to discover serum proteins that are associated with active disease in MS patients with the goal to determine if a panel of serum markers increases the sensitivity in identifying relapses compared to using only NfL levels.

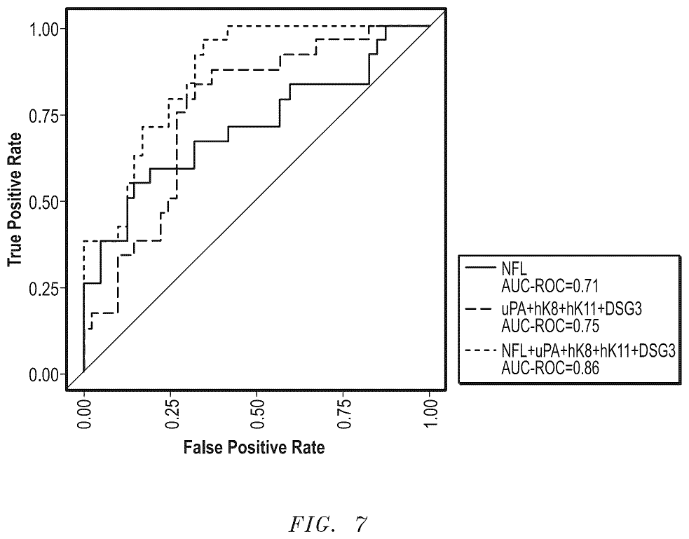

FIG. 7 is a graph that shows the AUCs for the various combinations.

Table 1. Summarizes the results in FIG. 7.

| Models | AUC |
|---|---|
| hK8 | 0.58 |
| DSG3 | 0.6 |
| hK11 | 0.67 |
| NFL | 0.71 |
| uPA | 0.76 |
| uPA + hK8 + hK11 + DSG3 | 0.75 |
| NFL + uPA + hK8 + hK11 + DSG3 | 0.86 |

An important metric used to measure performance of a classification model is called area under the ROC curve (AUC) [32]. In this study, ROC curves for three classification models were compared: 1. All proteins measured, 2. NfL alone and 3. NfL with uPA, hK8, hK11, DSG3. It was found that a ROC curve using NfL alone had an AUC of 0.71 to classify relapse, which is similar to an AUC of 0.663 that was reported previously for NfL [31]. When the other four proteins together with NfL were used, the classification model was more accurate at classifying a relapse (with an AUC of 0.87) than using NfL alone (FIG. 6). These data demonstrate that a panel of biomarkers classifies relapse with a higher accuracy than NfL alone.

In this cross-sectional discovery cohort, the inventors tried to minimize factors that could confound the discovery of markers of acute relapse. The inventors limited confounders by recruiting RRMS patients early after their diagnosis and before they were put on disease-modifying therapies (DMTs) and by correcting for age and sex during this analysis. The inventors also considered the effect of corticosteroid treatment on biomarker discovery. High-dose intravenous or oral steroids are used as a fast-acting treatment for MS patients who are experiencing disease exacerbations [33, 34]. In the discovery cohort, about 50% of the relapsing patients were given steroids within 4 weeks prior to the time of blood draw. It was found that patients on steroids had altered levels of blood markers. There were two protein markers of interest that were elevated in the steroid patients, FKBP5 protein and NfL. Previous studies have identified that FKBP5 expression is directly induced by steroid, where there is a dose dependent increase in transcription of this gene by in vitro steroid treatment [30]. In this cohort it was found that levels of FKBP5 were elevated in relapsing MS patients on steroid treatment compared to relapsing MS patients not on steroid treatment in the cross-sectional cohort of RRMS patients. Strikingly, FKBP5 levels significantly decreased with time after stopping steroids in the relapsing MS patients, which provides evidence that steroids are directly affecting levels of biomarkers. The inventors also found that NfL levels were elevated in patients on steroids compared to patients not on steroids. However, in this case, NfL did not significantly decrease with time after steroid treatment stoppage. Thus, it may be that steroid treatment does not cause elevated NfL, but rather, that the high NfL levels reflect the severity of relapse in these patients.

Next, the inventors determined if any disease activity biomarkers identified in the cross-sectional cohort could be verified in a second independent cohort. This second cohort was a longitudinal study of 12 patients where blood specimens were first taken at the time of an acute relapse and a second specimen was taken during remission within 12 months of the relapse. This longitudinal cohort differed from the discovery cohort in two ways. In the first cohort, the blood samples were serum, and samples from the second cohort were plasma. Both serum and plasma are derived from blood and commonly used in clinical and biological studies. After blood has coagulated, fibrin clots, blood cells and other coagulation-related factors are removed and produce serum. Plasma, on the other hand, is obtained when blood is treated with an anti-coagulant like EDTA or heparin before removal of blood cells. Due to these differences in how the samples are processed, previous studies have reported differences in measurement of cytokines and chemokines in serum and plasma studies [40, 41]. Second, there were also differences in DMTs that the patients received between the two cohorts. The patients from the first cohort were treatment naïve at the time of blood draw. However, patients from the second cohort were on various treatments when blood was drawn including glatiramer acetate, fingolimod, natalizumab, dimethyl fumarate and ocrelizumab. Multiple studies have previously shown that administration of DMTs changes the blood protein profiles of RRMS patients [35, 36]. Despite these differences in sample type and treatments between the two cohorts, the inventors identified 5 common proteins that were significantly different in relapse and remission in RRMS patients in both cohorts. These proteins were NfL, uPA, hK8, hK11 and DSG3.

The biological significance of the alteration of these proteins during relapse and remission in patients has not been fully elucidated. However, the established function of these molecules provides clues to their effects in MS. NfL has been shown to be elevated in the blood of relapsing MS patients and is considered a marker neuronal damage in MS. Urokinase-type plasminogen activator (uPA) is a serine-protease that converts plasminogen to plasmin and is mainly expressed by neutrophils, monocytes, macrophages and activated T-cells [37]. Plasminogen activators play an important role in the clearance of fibrin/fibrinogen deposits from sites of inflammation, thus preventing further inflammatory activity. In this study, lower levels of uPA during a relapse compared to remission were found.

The inventors also found lower levels of hK8 and hK11, members of the kallikrein family of proteases, during relapse in patients. Kallikreins, specifically KLKI and KLK6, have been found to be associated with secondary progressive MS [39]. The role of kallikreins, hK8 and hK11, during relapses in MS is unknown. Finally, lower levels of desmoglein-3 (DSG3) were found during relapse. DSG3 which is a component of intercellular desmosome junctions is important for maintaining tight junctions in mucosal epithelial barriers in the intestines [40]. Therefore, dysregulation of DSG3 could be an indicator of intestinal permeability, which is a biological process that has been linked to MS pathology [41-43].

The present inventors discovered novel blood-based biomarkers for identifying and monitoring relapses in MS, and treating MS. The data highlight the importance of identifying confounding factors that influence biomarker discovery, including age, sex, treatment, and blood sample type. Finally, a panel was identified of biomarkers that increased the accuracy of identifying relapse compared to using NfL alone.

Thus, the present disclosure relates to a method of treating a subject having relapsing-remitting multiple sclerosis (RRMS) that is undergoing relapse, comprising, consisting essentially of, or consisting of: (a) determining a level of expression for two or more biomarkers selected from urokinase plasminogen activator (uPA), kallikrein-8 (hK8), kallikrein-11 (hK11), or desmoglein-3 (DSG3) in a biological sample of a subject when compared to the same type of sample from a subject or a population of subjects that do not have RRMS; (b) diagnosing the subject in (a) as undergoing relapse if the level of expression of the two or more biomarkers has decreased in the subject; and (c) administering a therapeutically effective amount of a treatment for RRMS to the subject diagnosed in (b). In one aspect, the biomarkers are uPA, hK8, hK11, or DSG3. In another aspect, the method further comprises determining the level of expression for NfL, uPA, hK8, hK11, or DSG3, wherein a combination of biomarkers reaches a higher area under the curve when compared to NfL alone. In another aspect, the therapeutic agent is selected from at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH) or ponesimod. In another aspect, the biological sample is selected from the group consisting of peripheral blood, plasma, serum, peripheral blood mononuclear cells (PBMCs), or cerebrospinal fluid (CSF). In another aspect, the relapse is acute phase relapse. In another aspect, the relapse is from a patient that had previously received at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod. In another aspect, the biomarkers are nucleic acid biomarkers, protein biomarkers, or combinations thereof. In another aspect, the method further comprises detecting in a sample from a subject with RRMS previously treated with a steroid treatment an increase in a level of expression of FK506 binding protein 1 (FKBP5), wherein cessation of steroid treatment causes a decrease in the level of expression of FKBP5.

The present disclosure also relates to a method for determining whether a subject with relapsing-remitting multiple sclerosis (RRMS) with an elevated level of an NfL biomarker is having or will have a relapse, comprising, consisting essentially of, or consisting of: (a) determining in a biological sample from the subject a level of expression for one or more biomarkers selected from uPA, hK8, hK11, or DSG3 when compared to the same sample from a subject or a population of subjects that do not have RRMS, wherein the combination of the NfL biomarker and the one or more biomarker selected from uPA, hK8, hK11, or DSG3 has a higher sensitivity and selectivity that using NfL biomarker alone; (b) diagnosing the subject in (a) as undergoing relapse if the expression level of the uPA, hK8, hK11 or DSG3 has decreased, and (c) administering a therapeutically effective amount of a treatment for RRMS to the subject diagnosed in (b). In one aspect, the method comprises selecting 2, 3, or 4 of the biomarkers selected from uPA, hK8, hK11, or DSG3. In another aspect, the method of claim 10, further comprising determining the level of expression for NfL, uPA, hK8, hK11, or DSG3 and calculating an area under the curve, wherein a combination of biomarkers reaches an area under the curve of at least 0.87. In another aspect, the therapeutic agent is selected from at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod. In another aspect, the biological sample is selected from the group consisting of peripheral blood, plasma, serum, peripheral blood mononuclear cells (PBMCs) or CSF. In another aspect, the relapse is acute phase relapse. In another aspect, the relapse is from a patient that had previously received at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod. In another aspect, the biomarkers are nucleic acid biomarkers, protein biomarkers, or combinations thereof. In another aspect, the method further comprises detecting in a sample from a subject with RRMS previously treated with a steroid treatment an increase in a level of expression of FK506 binding protein 1 (FKBP5), wherein cessation of steroid treatment causes a decrease in the level of expression of FKBP5.

Also, the present disclosure relates to a method for detecting relapse in a subject with relapsing-remitting multiple sclerosis (RRMS), comprising, consisting essentially of, or consisting of: (a) determining a level of expression for two or more biomarkers selected from uPA, hK8, hK11, or DSG3 in a biological sample of the subject when compared to the same sample from a subject or a population of subjects that do not have RRMS and wherein one of the biomarkers is not an NfL biomarker, wherein the two or more biomarker selected from uPA, hK8, hK11, or DSG3 has a higher sensitivity and selectivity that the Nfl biomarker alone; (b) diagnosing the subject in (a) as undergoing relapse if the level of expression of the two or more biomarkers has decreased in the subject, and (c) administering a therapeutically effective amount of a treatment for RRMS to the subject diagnosed in (b). In one aspect, the method comprises selecting 2, 3, or 4 of the biomarkers selected from uPA, hK8, hK11, or DSG3. In another aspect, the therapeutic agent is selected from at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod. In another aspect, the biological sample is selected from the group consisting of peripheral blood, plasma, serum, peripheral blood mononuclear cells (PBMCs), or CSF. In another aspect, the relapse is acute phase relapse. In another aspect, the relapse is from a patient that had previously received at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod, and selecting a new therapy. In another aspect, the biomarkers are nucleic acid biomarkers, protein biomarkers, or combinations thereof. In another aspect, the sample from the subject with RRMS has been previously detected to have a higher level of NfL expression as compared to the sample from subjects that do not have RRMS. In another aspect, the method further comprises detecting in a sample from a subject with RRMS previously treated with a steroid treatment an increase in a level of expression of FK506 binding protein 1 (FKBP5), wherein cessation of steroid treatment causes a decrease in the level of expression of FKBP5.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Hafler, D. A., Multiple sclerosis. J Clin Invest, 2004. 113 (6): p. 788-94.
2. Mills, E. A., A. Mirza, and Y. Mao-Draayer, Emerging Approaches for Validating and Managing Multiple Sclerosis Relapse. Front Neurol, 2017. 8: p. 116.
3. Avasarala, J., Redefining Acute Relapses in Multiple Sclerosis: Implications for Phase 3 Clinical Trials and Treatment Algorithms. Innov Clin Neurosci, 2017. 14 (3-4): p. 38-40.
4. Zivadinov, R., et al., Cumulative gadodiamide administration leads to brain gadolinium deposition in early MS. Neurology, 2019. 93 (6): p. e611-e623.
5. Kuhle, J., et al., Serum neurofilament light chain in early relapsing remitting MS is increased and correlates with CSF levels and with MRI measures of disease severity. Multiple Sclerosis Journal, 2016. 22 (12): p. 1550-1559.
6. Thebault, S., et al., Serum neurofilament light in MS: The first true blood-based biomarker? Mult Scler, 2021: p. 1352458521993066.
7. Disanto, G., et al., Serum Neurofilament light: A biomarker of neuronal damage in multiple sclerosis. Ann Neurol, 2017. 81 (6): p. 857-870.
8. Varhaug, K. N., et al., Neurofilament light chain predicts disease activity in relapsing-remitting MS. Neurol Neuroimmunol Neuroinflamm, 2018. 5 (1): p. e422.
9. Ashton, N. J., et al., A multicentre validation study of the diagnostic value of plasma neurofilament light. Nat Commun, 2021. 12 (1): p. 3400.
10. Delaby, C., et al., Differential levels of Neurofilament Light protein in cerebrospinal fluid in patients with a wide range of neurodegenerative disorders. Sci Rep, 2020. 10 (1): p. 9161.
11. Khalil, M., et al., Serum neurofilament light levels in normal aging and their association with morphologic brain changes. Nature Communications, 2020. 11 (1).
12. Myhr, K. M. and O. Torkildsen, Serum NFL levels should be used to monitor multiple sclerosis evolution-No. Multiple Sclerosis Journal, 2020. 26 (1): p. 19-21.
13. Thompson, A. J., et al., Diagnosis of multiple sclerosis: 2017 revisions of the McDonald criteria. Lancet Neurol, 2018. 17 (2): p. 162-173.
14. Assarsson, E., et al., Homogenous 96-plex PEA immunoassay exhibiting high sensitivity, specificity, and excellent scalability. PLOS One, 2014. 9 (4): p. e95192.
15. Team, R. C., R: A language and environment for statistical computing. 2021, R Foundation for Statistical Computing: Vienna, Austria.
16. Ritchie, M. E., et al., limma powers differential expression analyses for RNA-sequencing and microarray studies. Nucleic Acids Res, 2015. 43 (7): p. e47.
17. Szklarczyk, D., et al., The STRING database in 2021: customizable protein-protein networks, and functional characterization of user-uploaded gene/measurement sets. Nucleic Acids Res, 2021. 49 (D1): p. D605-D612.
18. Kuhn, M., Building Predictive Models in R Using the caret Package. Journal of Statistical Software, 2008. 28 (5): p. 1-26.
19. John, C. R., MLeval: Machine Learning Model Evaluation. 2020, R package version 0.3.
20. Hegen, H., et al., Cytokine profiles show heterogeneity of interferon-beta response in multiple sclerosis patients. Neurol Neuroimmunol Neuroinflamm, 2016. 3 (2): p. e202.
21. Hartung, H. P., et al., Interleukin 17F level and interferon beta response in patients with multiple sclerosis. JAMA Neurol, 2013. 70 (8): p. 1017-21.
22. Agasing, A. M., et al., Transcriptomics and proteomics reveal a cooperation between interferon and T-helper 17 cells in neuromyelitis optica. Nat Commun, 2020. 11 (1): p. 2856.
23. Beck, R. W., et al., A randomized, controlled trial of corticosteroids in the treatment of acute optic neuritis. The Optic Neuritis Study Group. N Engl J Med, 1992. 326 (9): p. 581-8.
24. Morrow, S. A., et al., The bioavailability of IV methylprednisolone and oral prednisone in multiple sclerosis. Neurology, 2004. 63 (6): p. 1079-80.
25. Elovaara, I., et al., Methylprednisolone reduces adhesion molecules in blood and cerebrospinal fluid in patients with MS. Neurology, 1998. 51 (6): p. 1703-8.
26. Gelati, M., et al., Methylprednisolone acts on peripheral blood mononuclear cells and endothelium in inhibiting migration phenomena in patients with multiple sclerosis. Arch Neurol, 2002. 59 (5): p. 774-80.
27. Leussink, V. I., et al., High-dose methylprednisolone therapy in multiple sclerosis induces apoptosis in peripheral blood leukocytes. Arch Neurol, 2001. 58 (1): p. 91-7.
28. Wandinger, K. P., et al., Effect of high-dose methylprednisolone administration on immune functions in multiple sclerosis patients. Acta Neurol Scand, 1998. 97 (6): p. 359-65.
29. Binder, E. B., The role of FKBP5, a co-chaperone of the glucocorticoid receptor in the pathogenesis and therapy of affective and anxiety disorders. Psychoneuroendocrinology, 2009. 34 Suppl 1: p. S186-95.
30. Vermeer, H., et al., Glucocorticoid-induced increase in lymphocytic FKBP51 messenger ribonucleic acid expression: a potential marker for glucocorticoid sensitivity, potency, and bioavailability. J Clin Endocrinol Metab, 2003. 88 (1): p. 277-84.
31. Novakova, L., et al., Monitoring disease activity in multiple sclerosis using serum neurofilament light protein. Neurology, 2017. 89 (22): p. 2230-2237.
32. Lasko, T. A., et al., The use of receiver operating characteristic curves in biomedical informatics. J Biomed Inform, 2005. 38 (5): p. 404-15.
33. Ontaneda, D. and A. D. Rae-Grant, Management of acute exacerbations in multiple sclerosis. Ann Indian Acad Neurol, 2009. 12 (4): p. 264-72.
34. Smets, I., et al., Corticosteroids in the management of acute multiple sclerosis exacerbations. Acta Neurol Belg, 2017. 117 (3): p. 623-633.
35. Khademi, M., et al., The effects of natalizumab on inflammatory mediators in multiple sclerosis: prospects for treatment-sensitive biomarkers. Eur J Neurol, 2009. 16 (4): p. 528-36.
36. Adamczyk-Sowa, M., et al., Oxidative Modification of Blood Serum Proteins in Multiple Sclerosis after Interferon Beta and Melatonin Treatment. Oxid Med Cell Longev, 2017. 2017: p. 7905148.
37. Zhukovsky, C., et al., Urokinase, CX3CL1, CCL2, TRAIL and IL-18 induced by interferon-beta treatment. Acta Neurol Scand, 2021. 143 (6): p. 602-607.
38. Gur-Wahnon, D., et al., The plasminogen activator system: involvement in central nervous system inflammation and a potential site for therapeutic intervention. J Neuroinflammation, 2013. 10: p. 124.
39. Scarisbrick, I. A., et al., Kallikreins are associated with secondary progressive multiple sclerosis and promote neurodegeneration. Biol Chem, 2008. 389 (6): p. 739-45.
40. Ungewiss, H., et al., Desmoglein 2 regulates the intestinal epithelial barrier via p38 mitogen-activated protein kinase. Sci Rep, 2017. 7 (1): p. 6329.
41. Nouri, M., et al., Intestinal barrier dysfunction develops at the onset of experimental autoimmune encephalomyelitis, and can be induced by adoptive transfer of auto-reactive T cells. PLOS One, 2014. 9 (9): p. e106335.
42. Buscarinu, M. C., et al., Altered intestinal permeability in patients with relapsing-remitting multiple sclerosis: A pilot study. Mult Scler, 2017. 23 (3): p. 442-446.
43. Buscarinu, M. C., et al., Intestinal Permeability in Relapsing-Remitting Multiple Sclerosis. Neurotherapeutics, 2018. 15 (1): p. 68-74.
44. Mills E A, Mirza A and Mao-Draayer Y. Emerging Approaches for Validating and Managing Multiple Sclerosis Relapse. Front Neurol 2017; 8:116. 2017 Mar. 29. DOI: 10.3389/fneur.2017.00116.
45. Becker M, Latarche C, Roman E, et al. No prognostic value of routine cerebrospinal fluid biomarkers in a population-based cohort of 407 multiple sclerosis patients. BMC Neurol 2015; 15:79. 2015 May 13. DOI: 10.1186/s12883-015-0330-4.

What is claimed is:

1. A method of treating a subject having relapsing-remitting multiple sclerosis (RRMS) that is undergoing relapse, comprising:
(a) determining a level of expression for two or more biomarkers selected from urokinase plasminogen activator (uPA), kallikrein-8 (hK8), kallikrein-11 (hK11), or desmoglein-3 (DSG3) in a biological sample of a subject when compared to the same type of sample from a subject or a population of subjects that do not have RRMS;
(b) diagnosing the subject in (a) as undergoing relapse if the level of expression of the two or more biomarkers has decreased in the subject; and
(c) administering a therapeutically effective amount of a treatment for RRMS to the subject diagnosed in (b).

2. The method of claim 1, wherein the biomarkers are uPA, hK8, hK11, or DSG3.

3. The method of claim 1, further comprising determining the level of expression for NfL, uPA, hK8, hK11, or DSG3, wherein a combination of biomarkers reaches a higher area under the curve when compared to NfL alone.

4. The method of claim 1, wherein the therapeutic agent is selected from at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH) or ponesimod.

5. The method of claim 1, wherein the biological sample is selected from the group consisting of peripheral blood, plasma, serum, peripheral blood mononuclear cells (PBMCs), or cerebrospinal fluid (CSF).

6. The method of claim 1, wherein the relapse is acute phase relapse.

7. The method of claim 1, wherein the relapse is from a patient that had previously received at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod.

8. The method of claim 1, wherein the biomarkers are nucleic acid biomarkers, protein biomarkers, or combinations thereof.

9. The method of claim 1, further comprising detecting in a sample from a subject with RRMS previously treated with a steroid treatment an increase in a level of expression of FK506 binding protein 1 (FKBP5), wherein cessation of steroid treatment causes a decrease in the level of expression of FKBP5.

10. A method for determining whether a subject with relapsing-remitting multiple sclerosis (RRMS) with an elevated level of an NfL biomarker is having or will have a relapse, comprising:
(a) determining in a biological sample from the subject a level of expression for one or more biomarkers selected from uPA, hK8, hK11, or DSG3 when compared to the same sample from a subject or a population of subjects that do not have RRMS, wherein the combination of the NfL biomarker and the one or more biomarker selected from uPA, hK8, hK11, or DSG3 has a higher sensitivity and selectivity that using NfL biomarker alone;
(b) diagnosing the subject in (a) as undergoing relapse if the expression level of the uPA, hK8, hK11 or DSG3 has decreased, and
(c) administering a therapeutically effective amount of a treatment for RRMS to the subject diagnosed in (b).

11. The method of claim 10, comprising selecting 2, 3, or 4 of the biomarkers selected from uPA, hK8, hK11, or DSG3.

12. The method of claim 10, further comprising determining the level of expression for NfL, uPA, hK8, hK11, or DSG3 and calculating an area under the curve, wherein a combination of biomarkers reaches an area under the curve of at least 0.87.

13. The method of claim 10, wherein the therapeutic agent is selected from at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod.

14. The method of claim 10, wherein the biological sample is selected from the group consisting of peripheral blood, plasma, serum, peripheral blood mononuclear cells (PBMCs) or CSF.

15. The method of claim 10, wherein the relapse is acute phase relapse.

16. The method of claim 10, wherein the relapse is from a patient that had previously received at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod.

17. The method of claim 10, wherein the biomarkers are nucleic acid biomarkers, protein biomarkers, or combinations thereof.

18. The method of claim 10, further comprising detecting in a sample from a subject with RRMS previously treated with a steroid treatment an increase in a level of expression of FK506 binding protein 1 (FKBP5), wherein cessation of steroid treatment causes a decrease in the level of expression of FKBP5.

19. A method for detecting relapse in a subject with relapsing-remitting multiple sclerosis (RRMS), comprising:
(a) determining a level of expression for two or more biomarkers selected from uPA, hK8, hK11, or DSG3 in a biological sample of the subject when compared to the same sample from a subject or a population of subjects that do not have RRMS and wherein one of the biomarkers is not an NfL biomarker, wherein the two or more biomarker selected from uPA, hK8, hK11, or DSG3 has a higher sensitivity and selectivity that the Nfl biomarker alone;
(b) diagnosing the subject in (a) as undergoing relapse if the level of expression of the two or more biomarkers has decreased in the subject, and
(c) administering a therapeutically effective amount of a treatment for RRMS to the subject diagnosed in (b).

20. The method of claim 19, comprising selecting 2, 3, or 4 of the biomarkers selected from uPA, hK8, hK11, or DSG3.

21. The method of claim 19, wherein the therapeutic agent is selected from at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod.

22. The method of claim 19, wherein the biological sample is selected from the group consisting of peripheral blood, plasma, serum, peripheral blood mononuclear cells (PBMCs), or CSF.

23. The method of claim 19, wherein the relapse is acute phase relapse.

24. The method of claim 19, wherein the relapse is from a patient that had previously received at least one of: glatiramer acetate (GA), beta-interferons, mitoxantrone, monomethyl fumarate, ozanimod, diroximel fumarate, cladribine, siponimod, ocrelizumab, daclizumab, alemtuzumab, PEG-interferon beta-la, dimethyl fumarate, teriflunomide, fingolimod, natalizumab, laquinimod, ofatumumab, ublituximab, steroids, adrenocorticotropic hormone (ACTH), or ponesimod, and selecting a new therapy.

25. The method of claim 19, wherein the biomarkers are nucleic acid biomarkers, protein biomarkers, or combinations thereof.

26. The method of claim 19, wherein the sample from the subject with RRMS has been previously detected to have a higher level of NfL expression as compared to the sample from subjects that do not have RRMS.

27. The method of claim 19, further comprising detecting in a sample from a subject with RRMS previously treated with a steroid treatment an increase in a level of expression of FK506 binding protein 1 (FKBP5), wherein cessation of steroid treatment causes a decrease in the level of expression of FKBP5.
